(19) United States
(12) Patent Application Publication
(10) Pub. No.: US 2025/0238909 A1
(43) Pub. Date: Jul. 24, 2025

(54) SYSTEMS AND METHODS FOR CORRECTING DISTORTION CAUSED BY OPTICAL ELEMENTS OF AN IMAGE VIEWER OF A COMPUTER-ASSISTED SURGICAL SYSTEM

(71) Applicant: Intuitive Surgical Operations, Inc., Sunnyvale, CA (US)

(72) Inventors: Adam Samaniego, San Carlos, CA (US); Clinton Carlisle, Sunnyvale, CA (US); Ian E. McDowall, Woodside, CA (US)

(21) Appl. No.: 19/033,248

(22) Filed: Jan. 21, 2025

Related U.S. Application Data
(60) Provisional application No. 63/623,656, filed on Jan. 22, 2024.

(51) Int. Cl. G06T 5/80; A61B 34/20

(52) U.S. Cl. CPC G06T 5/80; A61B 34/20

(57) ABSTRACT

An illustrative system includes one or more processors configured to perform a process. The process may comprise obtaining a distortion factor representative of an amount of distortion imparted by one or more optical elements of an image viewer of a computer-assisted surgical system on images displayed by way of the image viewer; obtaining image data representative of an image that is displayable by way of the image viewer; modifying, based on the distortion factor, the image data to correct for the distortion imparted by the one or more optical elements of the image viewer, the modifying resulting in modified image data representative of the image; and directing the computer-assisted surgical system to display, based on the modified image data, the image by way of the image viewer.

### RELATED APPLICATIONS

The present application claims priority under 35 U.S.C. § 119(e) to U.S. Provisional Patent Application No. 63/623,656, filed on Jan. 22, 2024, which is incorporated herein by reference in its entirety.

### BACKGROUND INFORMATION

A computer-assisted surgical system that employs robotic and/or teleoperation technology typically includes a stereoscopic image viewer configured to provide, for display to a surgeon, images of an imaging space (e.g., a surgical space) as captured by an imaging device such as an endoscope. While the surgeon's eyes are positioned in front of viewing lenses of the stereoscopic image viewer, the surgeon may view the images of the surgical space while remotely manipulating one or more surgical instruments located within the surgical space. The surgical instruments are attached to one or more manipulator arms of a surgical instrument manipulating system included as part of the computer-assisted surgical system.

The stereoscopic image viewer of a computer-assisted surgical system has to display a pair of images (one to each eye) which are precisely overlapped. In addition, a compact stereoscopic image viewer typically involves using one or more optical elements that apply optical power to locate an image-render plane at a specified distance from the user. However, such optical elements may create distortion (e.g., barrel distortion, pincushion distortion, etc.) of the displayed images for each eye. This distortion inhibits overlapping the pair of images. If not corrected, such distortion can degrade the user's experience (e.g., by causing nausea, headaches, etc.) while the user operates the computer-assisted surgical system. To correct this distortion, some computer-assisted surgical systems include distortion-correction lens elements placed close to a flat panel display of the stereoscopic image viewer. However, such distortion-correction lens elements are complex, large, costly, and difficult to manufacture/source. Accordingly, there remains room to improve distortion correction in an image viewer of a computer-assisted surgical system.

### SUMMARY

An example system comprises one or more processors configured to perform a process comprising: obtaining a distortion factor representative of an amount of distortion imparted by one or more optical elements of an image viewer of a computer-assisted surgical system on images displayed by way of the image viewer; obtaining image data representative of an image that is displayable by way of the image viewer; modifying, based on the distortion factor, the image data to correct for the distortion imparted by the one or more optical elements of the image viewer, the modifying resulting in modified image data representative of the image; and directing the computer-assisted surgical system to display, based on the modified image data, the image by way of the image viewer.

An example computer-assisted surgical system comprises an image viewer including: a display device; and an optical assembly through which a user views images displayed by the display device, the optical assembly provided along an optical path between the display device and an eye of the user, the optical assembly imparting a distortion on images displayed by the display device when viewed through the optical assembly; wherein the display device is configured to: receive images to be displayed by way of the image viewer; and display the images based on a distortion factor that corrects for the distortion imparted by the optical assembly.

An additional example computer-assisted surgical system comprises one or more manipulator arms; and one or more processors configured to perform a process comprising: obtaining a distortion factor representative of an amount of distortion imparted by one or more optical elements of an image viewer; obtaining image data representative of an image that is displayable by way of the image viewer; modifying, based on the distortion factor, the image data to correct for the distortion imparted by the one or more optical elements of the image viewer, the modifying resulting in modified image data representative of the image; and presenting, based on the modified image data, the image by way of the image viewer.

An example method comprises obtaining, by an image processing system, a distortion factor representative of an amount of distortion imparted by one or more optical elements of an image viewer of a computer-assisted surgical system on images displayed by way of the image viewer; obtaining, by the image processing system, image data representative of an image that is displayable by way of the image viewer; modifying, by the image processing system and based on the distortion factor, the image data to correct for the distortion imparted by the one or more optical elements of the image viewer, the modifying resulting in modified image data representative of the image; and directing, by the image processing system, the computer-assisted surgical system to display, based on the modified image data, the image by way of the image viewer.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an example computer-assisted surgical system according to principles described herein.

FIG. 2 illustrates an example implementation of an image viewer configured according to principles described herein.

FIG. 3 illustrates an example image processing system according to principles described herein.

FIG. 4 illustrates an example flow chart depicting various operations that may be performed by the image processing system illustrated in FIG. 3 according to principles described herein.

FIG. 5 illustrates an example view of a modified image that has been modified based on a distortion factor according to principles described herein.

FIG. 6 illustrates a simplified version of a display panel configured to correct for distortion according to principles described herein.

FIG. 7 illustrates an example method for correcting distortion caused by optical elements of an image viewer of a computer-assisted surgical system according to principles described herein.

FIG. 8 illustrates an example computing device according to principles described herein.

### DETAILED DESCRIPTION

Systems and methods for correcting distortion caused by optical elements of an image viewer of a computer-assisted surgical system are described herein. As will be described in more detail below, an illustrative system includes one or more processors configured to perform a process. The process may comprise obtaining a distortion factor representative of an amount of distortion imparted by one or more optical elements of an image viewer of a computer-assisted surgical system on images displayed by way of the image viewer, obtaining image data representative of an image that is displayable by way of the image viewer, modifying, based on the distortion factor, the image data to correct for the distortion imparted by the one or more optical elements of the image viewer, the modifying resulting in modified image data representative of the image, and directing the computer-assisted surgical system to display, based on the modified image data, the image by way of the image viewer.

Various advantages and benefits are associated with systems and methods described herein. For example, systems and methods such as those described herein may facilitate correcting distortion caused by one or more optical elements of an image viewer in a manner that improves user experience, is more cost effective, and/or is more efficient than conventional image viewers. For example, systems and methods such as those described herein do not require the use of costly and difficult to manufacture distortion-correction lenses. In addition, by eliminating the need for such distortion-correction lenses, it is possible to reduce or eliminate undesired stray light paths in the image viewer. These and other benefits that may be realized by the systems and methods described herein will be evident from the disclosure that follows.

Example systems described herein may be configured to operate as part of or in conjunction with a plurality of different types of computer-assisted surgical systems. The different types of computer-assisted surgical systems may include any type of computer-assisted surgical system as may serve a particular implementation, such as a computer-assisted surgical system designed for use in minimally-invasive medical procedures, for example. In certain examples, a type of computer-assisted surgical system may include a system in which one or more surgical devices (e.g., surgical instruments) are manually (e.g., laparoscopically) controlled by a user. In certain examples, a type of computer-assisted surgical system may include a robotic surgical system configured to facilitate operation of one or more smart instruments (e.g., smart sub-surface imaging devices) that may be manually and/or robotically controlled by a user. In certain implementations, the plurality of different types of computer-assisted surgical systems may be of different types at least because they include different types of surgical instrument manipulating systems. For example, a first computer-assisted surgical system may include a first type of surgical instrument manipulating system, a second computer-assisted surgical system may include a second type of surgical instrument manipulating system, and a third computer-assisted surgical system may include a third type of surgical instrument manipulating system.

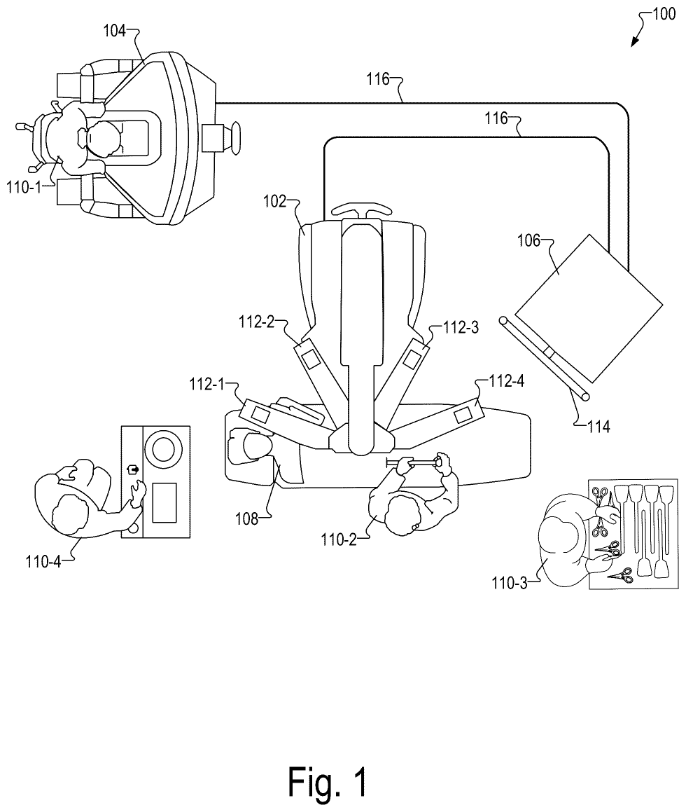

Each type of surgical instrument manipulating system may have a different architecture (e.g., a manipulator arm architecture), have a different kinematic profile, and/or operate according to different configuration parameters. An illustrative computer-assisted surgical system with a first type of surgical instrument manipulating system will now be described with reference to FIG. 1. The described computer-assisted surgical system is illustrative and not limiting. Systems such as those described herein may operate as part of or in conjunction with the described computer-assisted surgical system and/or any other suitable computer-assisted surgical system.

FIG. 1 illustrates an example computer-assisted surgical system 100 (“surgical system 100”). As shown, surgical system 100 includes a surgical instrument manipulating system 102 (“manipulating system 102”), a user control system 104, and an auxiliary system 106 communicatively coupled one to another. Additional or alternative components may be included in surgical system 100 as may serve a particular implementation.

Surgical system 100 may be utilized by a surgical team to perform a computer-assisted surgical procedure on a patient 108. As shown, the surgical team may include a surgeon 110-1, an assistant 110-2, a nurse 110-3, and an anesthesiologist 110-4, all of whom may be collectively referred to as “surgical team members 110.” Additional or alternative surgical team members may be present during a surgical session as may serve a particular implementation.

While FIG. 1 illustrates an ongoing minimally invasive surgical procedure, surgical system 100 may similarly be used to perform open surgical procedures or other types of surgical procedures that may similarly benefit from the accuracy and convenience of surgical system 100. Additionally, it will be understood that the surgical session throughout which surgical system 100 may be employed may not only include an operative phase of a surgical procedure, as is illustrated in FIG. 1, but may also include preoperative, postoperative, and/or other suitable phases of the surgical procedure. A surgical procedure may include any procedure in which manual and/or instrumental techniques (e.g., teleoperated instrumental techniques) are used on a patient to investigate, diagnose, or treat a physical condition of the patient. Additionally, a surgical procedure may include any procedure that is not performed on a live patient, such as a calibration procedure, a simulated training procedure, and an experimental or research procedure.

As shown in FIG. 1, surgical instrument manipulating system 102 includes a plurality of manipulator arms 112 (e.g., manipulator arms 112-1 through 112-4) to which a plurality of robotic surgical instruments (“robotic instruments”) (not shown) may be coupled. As used herein, a “robotic instrument” refers to any instrument that may be directly attached to (e.g., plugged into, fixedly coupled to, mated to, etc.) a manipulator arm (e.g., manipulator arm 112-1) such that movement of the manipulator arm directly causes movement of the instrument. Each robotic instrument may be implemented by any suitable therapeutic instrument (e.g., a tool having tissue-interaction functions), imaging device (e.g., an endoscope), diagnostic instrument, or the like that may be used for a computer-assisted surgical procedure (e.g., by being at least partially inserted into patient 108 and manipulated to perform a computer-assisted surgical procedure on patient 108). In some examples, one or more of the robotic instruments includes force-sensing and/or other sensing capabilities.

In the example shown in FIG. 1, manipulator arms 112 of manipulating system 102 are attached on a distal end of an overhead boom that extends horizontally. However, manipulator arms 112 may have other configurations in certain implementations. In addition, while manipulating system 102 is depicted and described herein as including four manipulator arms 112, it will be recognized that manipulating system 102 may include only a single manipulator arm 112 or any other number of manipulator arms as may serve a particular implementation.

Manipulator arms 112 and/or robotic instruments attached to manipulator arms 112 may include one or more displacement transducers, orientational sensors, and/or positional sensors (hereinafter “surgical system sensors”) used to generate raw (e.g., uncorrected) kinematics information. One or more components of surgical system 100 may be configured to use the kinematics information to track (e.g., determine positions of) and/or control the robotic instruments.

In addition, manipulator arms 112 may each include or otherwise be associated with a plurality of motors or actuators that control movement of manipulator arms 112 and/or the surgical instruments attached thereto. For example, manipulator arm 112-1 may include or otherwise be associated with a first internal motor (not explicitly shown) configured to yaw manipulator arm 112-1 about a yaw axis. In like manner, manipulator arm 112-1 may be associated with a second internal motor (not explicitly shown) configured to drive and pitch manipulator arm 112-1 about a pitch axis. Likewise, manipulator arm 112-1 may be associated with a third internal motor (not explicitly shown) configured to slide manipulator arm 112-1 along insertion axis. Manipulator arms 112 may each include a drive train system driven by one or more of these motors in order to control the pivoting of manipulator arms 112 in any manner as may serve a particular implementation. As such, if a robotic instrument attached, for example, to manipulator arm 112-1 is to be mechanically moved, one or more of the motors coupled to the drive train may be energized to move manipulator arm 112-1.

Robotic instruments attached to manipulator arms 112 may each be positioned in an imaging space. An “imaging space” as used herein may refer to any space or location where an imaging operation may be performed by an imaging device such as described herein. In certain examples, an imaging space may correspond to a surgical space. A “surgical space” may, in certain examples, be entirely disposed within a patient and may include an area within the patient at or near where a surgical procedure is planned to be performed, is being performed, or has been performed. For example, for a minimally invasive surgical procedure being performed on tissue internal to a patient, the surgical space may include the tissue, anatomy underlying the tissue, as well as space around the tissue where, for example, robotic instruments and/or other instruments being used to perform the surgical procedure are located. In other examples, a surgical space may be at least partially disposed external to the patient at or near where a surgical procedure is planned to be performed, is being performed, or has been performed on the patient. For instance, surgical system 100 may be used to perform an open surgical procedure such that part of the surgical space (e.g., tissue being operated on) is internal to the patient while another part of the surgical space (e.g., a space around the tissue where one or more instruments may be disposed) is external to the patient. A robotic instrument may be referred to as being positioned or located at or within a surgical space when at least a portion of the robotic instrument (e.g., a distal portion of the robotic instrument) is located within the surgical space. Example imaging spaces and/or images of imaging spaces will be described herein.

User control system 104 is configured to facilitate control by surgeon 110-1 of manipulator arms 112 and robotic instruments attached to manipulator arms 112. For example, surgeon 110-1 may interact with user control system 104 to remotely move, manipulate, or otherwise teleoperate manipulator arms 112 and the robotic instruments. To this end, user control system 104 may provide surgeon 110-1 with one or more images of a surgical space associated with patient 108 as captured by an imaging device. In certain examples, user control system 104 may include a stereoscopic image viewer having two displays where stereoscopic image pairs of a surgical space associated with patient 108 and generated by a stereoscopic imaging system may be viewed by surgeon 110-1. Surgeon 110-1 may utilize the image or images to perform one or more procedures with one or more robotic instruments attached to manipulator arms 112.

To facilitate control of robotic instruments, user control system 104 may include a set of master controls (not shown). These master controls may be manipulated by surgeon 110-1 to control movement of robotic instruments (e.g., by utilizing robotic and/or teleoperation technology). The master controls may be configured to detect a wide variety of hand, wrist, and finger movements by surgeon 110-1. In this manner, surgeon 110-1 may intuitively perform a surgical procedure using one or more robotic instruments.

In some examples, user control system 104 may be further configured to facilitate control by surgeon 110-1 of other components of surgical system 100. For example, surgeon 110-1 may interact with user control system 104 to change a configuration or operating mode of surgical system 100, to change a display mode of surgical system 100, to generate additional control signals used to control surgical instruments attached to manipulator arms 112, to facilitate switching control from one robotic instrument to another, to facilitate interaction with other instruments and/or objects within the surgical space, or to perform any other suitable operation. To this end, user control system 104 may also include one or more input devices (e.g., foot pedals, buttons, switches, etc.) configured to receive input from surgeon 110-1.

In some examples, auxiliary system 106 includes one or more computing devices configured to perform primary processing operations of surgical system 100. The one or more computing devices included in auxiliary system 106 may control and/or coordinate operations performed by various other components (e.g., manipulating system 102 and/or user control system 104) of surgical system 100. For example, a computing device included in user control system 104 may transmit instructions to manipulating system 102 by way of the one or more computing devices included in auxiliary system 106. As another example, auxiliary system 106 may receive, from manipulating system 102, and process image data representative of images captured by an imaging device attached to one of manipulator arms 112.

In some examples, auxiliary system 106 is configured to present visual content to surgical team members 110 who may not have access to the images provided to surgeon 110-1 at user control system 104. To this end, as shown, auxiliary system 106 includes a display monitor 114 configured to display one or more user interfaces, such as images of the surgical space, information associated with patient 108 and/or the surgical procedure, and/or any other visual content as may serve a particular implementation. For example, display monitor 114 may display images of the surgical space together with additional content (e.g., representations of target objects, graphical content, contextual information, etc.) concurrently displayed with the images. In some embodiments, display monitor 114 is implemented by a touchscreen display with which surgical team members 110 may interact (e.g., by way of touch gestures) to provide user input to surgical system 100.

Manipulating system 102, user control system 104, and auxiliary system 106 may be communicatively coupled one to another in any suitable manner. For example, as shown in FIG. 1, manipulating system 102, user control system 104, and auxiliary system 106 are communicatively coupled by way of control lines 116, which may represent any wired or wireless communication link as may serve a particular implementation. To this end, manipulating system 102, user control system 104, and auxiliary system 106 may each include one or more wired or wireless communication interfaces, such as one or more local area network interfaces, Wi-Fi network interfaces, cellular interfaces, etc.

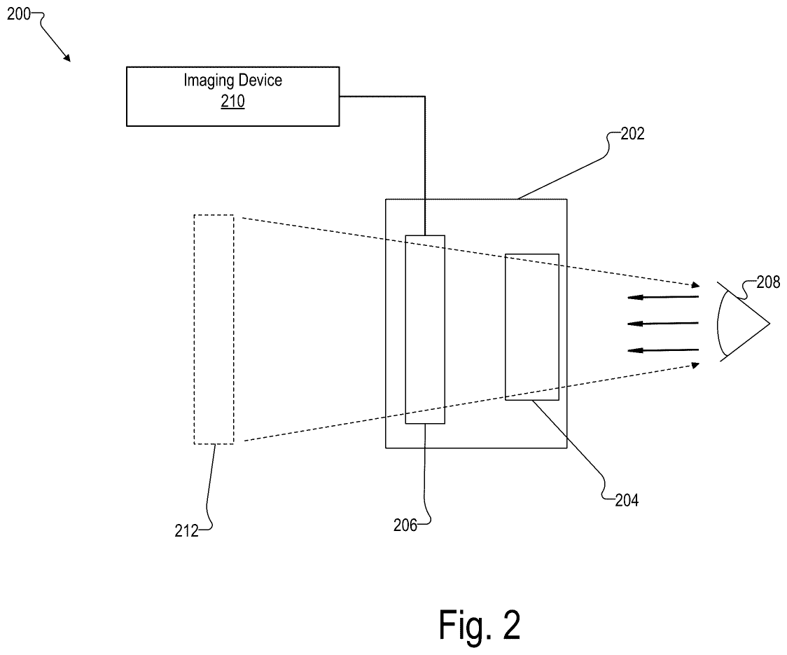

FIG. 2 illustrates an exemplary implementation 200 an image viewer 202 that may be configured according to principles described herein as part of a computer-assisted surgical system such as surgical system 100. As shown in FIG. 2, image viewer 202 includes an optical assembly 204, and a display device 206 arranged relative to one another along an optical path. Image viewer 202 may include additional or alternative components as may serve a particular implementation.

As shown in FIG. 2, optical assembly 204 is arranged to be in front of an eye 208 of a user such that images displayed by display device 206 are viewable by way of optical assembly 204. Although FIG. 2 only shows one optical assembly 204 and a display device 206, it is understood that an additional optical assembly and display device may be provided for an additional eye of the user.

The depiction of image viewer 202 in FIG. 2 is schematic to illustrate basic components of image viewer 202. As such, the relative size, orientation, and arrangement of optical assembly 204 and display device 206 are provided for illustrative purposes only. It is understood that the components shown in FIG. 2 may be configured differently in other implementations and/or may include any suitable additional elements. For example, in certain implementations, image viewer 202 may further include one or more reflectors provided along an optical path between display device 206 and optical assembly 204. In such examples, display device 206 may be provided in a side mounted position and a reflector may redirect an image displayed by display device 206 toward eye 208.

Image viewer 202 may be implemented in any suitable manner as part of the computer assisted surgical system. For example, image viewer 202 may be part of a surgeon console (e.g., user control system 104) that is communicatively coupled to one or more manipulator arms (e.g., manipulator arms 112) and an imaging device 210 communicatively coupled to a computer-assisted surgical system.

As shown in FIG. 2, optical assembly 204 is provided along an optical path between display device 206 and an eye 208 of a user. Optical assembly 204 may include one or more optical elements that impart a distortion on images displayed by display device 206 when viewed through optical assembly 204. Optical assembly 204 may include a single optical element or lens as shown in FIG. 2 or may include a lens group composed of a plurality of lenses or optical elements. For example, optical assembly 204 may include an eyepiece and one or more optical elements. Optical assembly 204 may include any suitable type of optical element as may serve a particular implementation. For example, optical assembly 204 may include a convex lens, a concave lens, a Frensel lens, and/or any other suitable type or combination of optical elements. Optical assembly 204 may also include any suitable additional optical imaging elements such as, for example, filters, gratings, etc.

Display device 206 may include any suitable display device as may serve a particular implementation. For example, in certain implementations display device 206 may correspond to a flat panel light-emitting diode (LED) display device, a liquid crystal display (LCD) device, a liquid crystal on silicon (LCOS) display device, a micro-electromechanical systems (MEMS) display device, a digital light processing (DLP) display device, organic light-emitting diode (OLED) display device, and/or any other suitable type of display device. In certain examples, display device 206 may correspond to a rectilinear display device. In certain alternative implementations, display device 206 may correspond to a non-rectilinear display device (e.g., a display device having a curved display architecture with a non-rectilinear pixel grid). Implementations using a non-rectilinear display device are described further herein.

Display device 206 is configured to receive images captured by imaging device 210 and display the images such that they are viewable along an optical path through optical assembly 204. Imaging device 210 may include any suitable type of imaging device as may serve a particular implementation. For example, imaging device 210 may be implemented by an endoscope that is engaged with a manipulator arm (e.g., manipulator arm 112-2) of the computer-assisted surgical system.

As shown in FIG. 2, eye 208 of a user may view an image displayed by display device 206 by way of optical assembly 204. Optical assembly 204 is configured to locate an image-render plane 212 at a specified distance from the user that is suitable for viewing. However, the image displayed by display device 206 may be distorted due to the optical power and/or other parameters associated with optical assembly 204 and/or image viewer 202. For example, such distortion may include a barrel distortion, a pincushion distortion, and/or any other type or combination of distortions. Other parameters that may affect the amount and/or type of distortion may include, for example, a focal length of optical assembly 204, a distance between display device 206 and optical assembly 204, a distance between eye 208 of the user and optical assembly 204, and/or any other suitable parameter.

As shown in FIG. 2, image viewer 202 does not include a distortion-correction lens that is specifically configured to correct distortion caused by optical assembly 204. In addition, image viewer 202 does not include a distortion correction lens included as part of optical assembly 204 that is configured to correct for distortion imparted by one or more other optical elements of optical assembly 204. Rather, display device 206 may be further configured to display images captured by imaging device 210 based on a distortion factor that corrects for the distortion imparted by optical assembly 204. As used herein, a “distortion factor” may be indicative of an amount of distortion imparted by one or more optical elements of an image viewer. The distortion factor may be due to intrinsic characteristics of optical assembly 204 and/or other parameters such as those described herein that may affect the amount of distortion.

Image viewer 202 may be configured in any suitable manner to correct for the distortion imparted by optical assembly 204. In certain examples, image viewer 202 may further include or otherwise be communicatively coupled to one or more processors configured to modify images captured by imaging device 210 based on the distortion factor. In such examples, distortion correction processing may be performed on an image to be displayed before the image is displayed by way of display device 206. Such distortion correction processing is configured to counteract the distortion caused by the optical assembly 204 such that, when the image is viewed by way of optical assembly 204, it does not appear distorted to the user.

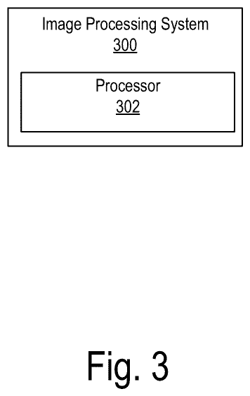

FIG. 3 shows an example image processing system 300 that may be implemented according to principles described herein to correct for distortion caused by one or more optical elements of an image viewer. As shown in FIG. 3, image processing system 300 (“system 300”) includes a processor 302. Processor 302 may be implemented as one or more processors of any suitable type as may serve a particular implementation. For example, in certain implementations, all or part of the functions or processes described herein may be implemented as special purpose logic circuitry (e.g., a field programmable gate array (FPGA) and/or an application-specific integrated circuit (ASIC).

System 300 may include additional or alternative elements as may serve a particular implementation. For example, system 300 may further include a memory selectively and communicatively coupled to processor 302. Such a memory and processor 302 may each include or be implemented by hardware and/or software components (e.g., processors, memories, communication interfaces, instructions stored in memory for execution by the processors, etc.). In some examples, such a memory and processor 302 may be implemented by a single device (e.g., a single computing device). In certain alternate examples a memory and processor 302 may be distributed between multiple devices and/or multiple locations as may serve a particular implementation.

In certain examples, a memory may maintain (e.g., store) executable data used by processor 302 to perform any of the operations described herein. For example, a memory may store instructions that may be executed by processor 302 to perform any of the operations described herein. Such instructions may be implemented by any suitable application, software, code, computer program, and/or other executable data instance.

A computer program may be written in any form of programming language including compiled and/or interpreted languages, and can be deployed in any form, including as a stand-alone program or as a module, component, subroutine, or other unit suitable for use in a computing environment. A computing program may be deployed to be executed by processor 302 at one site or distributed across multiple sites and interconnected by a network.

In certain examples, a memory may also maintain any data received, generated, managed, used, and/or transmitted by processor 302. For example, a memory may maintain any suitable data associated with correcting a distortion caused by one or more optical elements. Such data may include, but is not limited to, data associated with distortion factors, factors associated with use of an image viewer, image data (e.g., endoscopic images) of an imaging space, user interface content (e.g., graphical objects, notifications, etc.), and/or any other suitable data.

Processor 302 may be configured to perform (e.g., execute instructions stored in a memory) various processing operations associated with correcting a distortion caused by one or more optical elements. For example, processor 302 may modify, based on a distortion factor, image data to correct for distortion imparted by one or more optical elements of an image viewer, the modifying resulting in modified image data representative of the image. Processor 302 may further direct a computer-assisted surgical system to display, based on the modified image data, the image by way of the image viewer. These and other operations that may be performed by processor 302 are described herein.

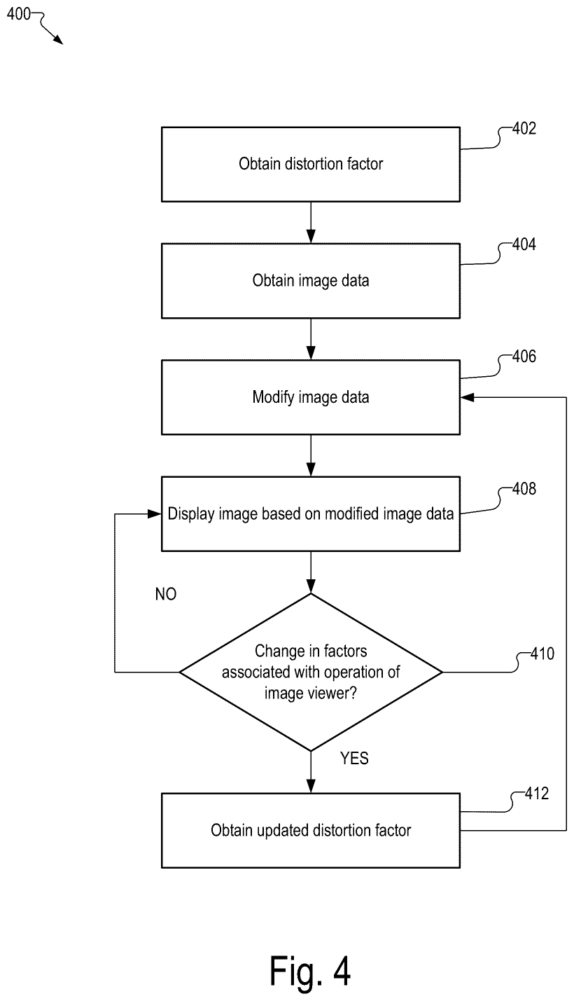

FIG. 4 illustrates a flow diagram 400 depicting various operations that may be performed by system 300 (e.g., processor 302) to correct for the distortion imparted by an optical assembly of an image viewer. One or more of the operations illustrated in FIG. 4 may be performed as part of an image processing pipeline performed by system 300.

At operation 402, system 300 obtains a distortion factor. This may be performed in any suitable manner. For example, system 300 may access the distortion factor from any suitable memory or storage device communicatively coupled to system 300. In certain examples, system 300 may additionally or alternatively obtain the distortion factor by accessing a pre-programmed distortion factor from logic of an FPGA.

In certain examples, system 300 may obtain the distortion factor by generating the distortion factor based on one or more factors or parameters associated with operation of an image viewer. For example, system 300 may generate the distortion factor based on the focal length of optical assembly 204, a distance between display device 206 and optical assembly 204, a distance between eye 208 of the user and optical assembly 204, an interpupillary distance between eye 208 and the other eye of the user, a distortion caused by imaging device 210, and/or any other suitable factor or parameter.

At operation 404, system 300 obtains image data representative of an image that is displayable by way of the image viewer. In certain examples, the image may be captured by an imaging device associated with a computer-assisted surgical system. This may be accomplished in any suitable manner. For example, system 300 may receive image data captured by imaging device 210 during a surgical procedure performed by the computer-assisted surgical system.

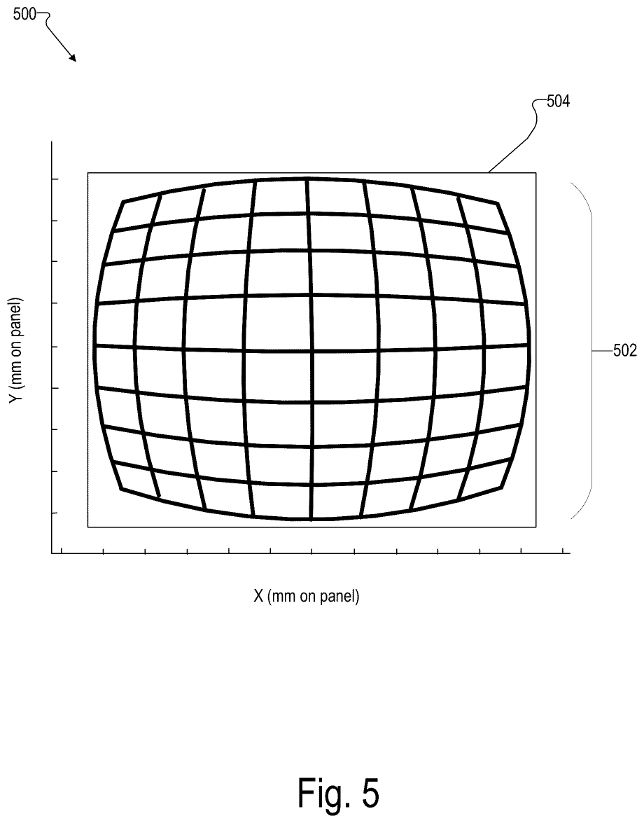

At operation 406, system 300 modifies, based on the distortion factor, the image data to correct for the distortion imparted by one or more optical elements of the image viewer. This may be accomplished in any suitable manner. For example, system 300 may pre-distort image data utilizing at least some pixels included in a plurality of pixels of the image. To illustrate, FIG. 5 shows an exemplary diagram 500 that depicts a modified image 502 in relation to a pixel area 504 of a display device. FIG. 5 illustrates an amount of distortion that may be applied to the image in certain examples. Pixel area 504 represents the dimensions that would typically be displayed without any distortion of the image data. Modified image 502 may include a repositioning and/or distorting of image data on a per pixel basis. This may be accomplished in any suitable manner. For example, the pixel grid of a display device may be mapped back to a source image and the image data may be interpolated from the source image. In such examples, system 300 may sample from the source image and interpolate whenever the image data does not perfectly align with a particular pixel location. As a result, when modified image 502 is viewed through an optical assembly of an image viewer, modified image 502 does not appear distorted to the user.

In certain examples, system 300 may modify the image data line by line for each pixel included in the image data.

In certain alternative examples, system 300 may modify the image data for only a subset of pixels included in a plurality of pixels of the image data. In such examples, there may be less distortion imparted on certain portions of an image viewed through an optical assembly than other portions. For example, pixels along a vertical center line of an image may either not appear distorted or may appear less distorted than pixels at the corners of an image. In such examples, system 300 may be configured to pre-distort pixel values for only a subset of pixels included in a plurality of pixels of an image. For example, the pre-distortion may only be applied to pixels that are positioned more than a pre-defined threshold distance from the vertical center line of an image.

In certain examples, system 300 may automatically switch from modifying each pixel to modifying only a subset of the pixels based on one or more factors associated with operation of a computer-assisted surgical system. For example, system 300 may automatically switch to modifying only a subset of the pixels based on bandwidth constraints, the type of surgical procedure (e.g., some types of surgical procedures may be less intricate and require less distortion), and/or any other suitable factor.

Returning to FIG. 4, at operation 408, system 300 directs a display device to display an image based on the modified image data. This may be accomplished in any suitable manner. For example, system 300 may direct the display device to display a modified image such as modified image 502 shown in FIG. 5 that uses less of pixel area 504.

In certain examples, one or more factors associated with operation of an image viewer may change in a manner that affects the amount of distortion and/or the type of distortion imparted on images viewed through an image viewer. For example, a distance between eye 208 of the user and optical assembly 204 may change during use by the user physically moving eye 208 away from image viewer 202. Such a change may result in more or less distortion being visible on images viewed through image viewer 202. Additionally or alternatively, the presence or absence of certain lenses (e.g., in a system with lenses swapped in and out) may result in a change that affects the amount of distortion. Additionally or alternatively, an optical element changing shape (e.g., deformable mirror, tuneable lens, etc.) may result in a change that affects the amount of distortion. Additionally or alternatively, a change in a thermal state of an image viewer may affect the amount of distortion. Additionally or alternatively, relative distances of elements in an optical assembly may change, which may affect the amount of distortion. Additionally or alternatively, where the user is looking in a surgical scene while using an image viewer may affect the amount of distortion that needs to be corrected. Accordingly, at operation 410, system 300 determines whether there has been a change in factors associated with operation of the image viewer. If the answer at operation 410 is “No,” the flow may return to operation 408. If the answer at operation 410 is “Yes,” system 300 may obtain an updated distortion factor at operation 412 that takes into consideration the change in the factors. The flow may then return to operation 406 where system 300 may modify the image data based on the updated distortion factor.

Operations 406 may be repeated any suitable number of times to ensure that distortion caused by one or more optical elements of an image viewer is dynamically corrected during operation of the image viewer. In certain examples, system 300 may continually monitor factors associated with operation of image viewer 202 to ensure that the distortion is corrected as the factors change during use.

In certain examples, system 300 may be configured to minimize the amount of time used to process the image data to correct for the distortion. For example, system 300 may process the image data such that a latency from image capture to displaying the image by way of the image viewer is less than 50 milliseconds.

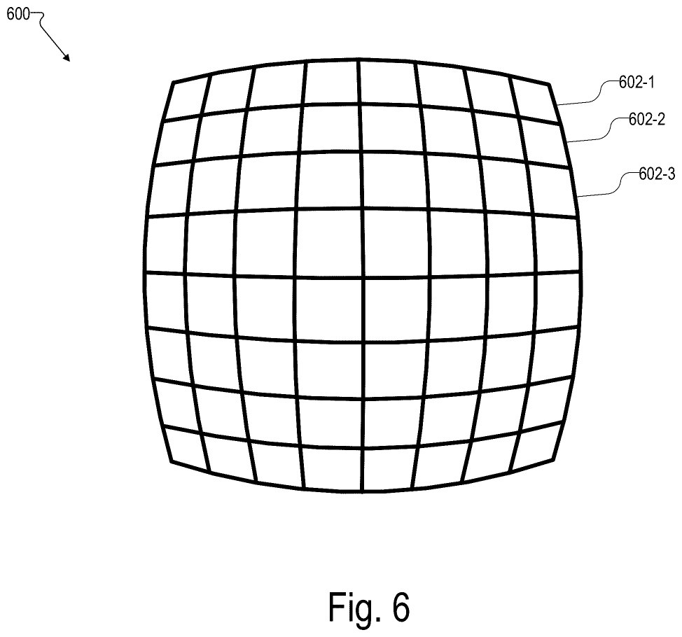

In certain additional or alternative examples, the distortion caused by an optical assembly of an image viewer and/or other components associated with an image viewer may be corrected by a hardware configuration of a display device (e.g., display device 206). In such examples, the display device may include pixels that are arranged in an array based on the distortion factor to correct for the distortion imparted by the optical assembly. This may be accomplished in any suitable manner. For example, an arrangement of the pixels of the display device may be non-rectilinear such that, when an image displayed by the display device is viewed by way of the optical elements of an image viewer, the image does not appear distorted to a user. To illustrate, FIG. 6 shows a simplified example of a display device 600 that includes a plurality of pixels 602 (e.g., pixels 602-1 through 602-3) that are arranged in a non-rectilinear manner. In the example shown in FIG. 6, the pixels are arranged in a barrel configuration that may facilitate correcting a pin cushion distortion that would otherwise be observed when viewing images by way of the optical assembly.

The number, shape, and/or arrangement of pixels 602 shown in FIG. 6 are provided for illustrative purposes only. It is understood that any suitable number and/or arrangement of pixels may be used as may serve a particular implementation. For example, the arrangement of pixels may be a non-rectilinear shape in certain implementations.

In certain additional or alternative examples, the distortion caused by an optical assembly of an image viewer and/or other components associated with an image viewer may be corrected in hardware by using a deformable optical device (deformable mirror, deformable lens, adjusting how a laser is scanned, etc.). System 300 may be configured in any suitable manner to control deformation of such a deformable device to correct for distortion in images viewable by way of an image viewer. For example, system 300 may dynamically control the deformation of a deformable element as one or more factors associated with an image viewer change during operation of the image viewer.

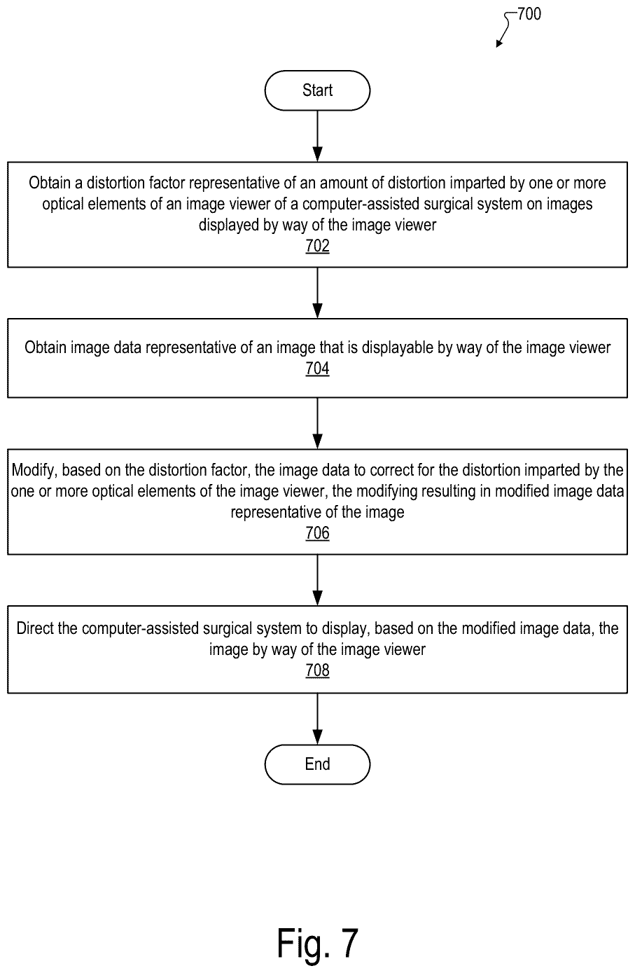

FIG. 7 illustrates an example method 700 for correcting distortion caused by optical elements of an image viewer of a computer-assisted surgical system. While FIG. 7 illustrates example operations according to one embodiment, other embodiments may omit, add to, reorder, and/or modify any of the operations shown in FIG. 7. One or more of the operations shown in FIG. 7 may be performed by a system such as system 300, any components included therein, and/or any implementation thereof.

At operation 702, an image processing system (e.g., image processing system 300) obtains a distortion factor representative of an amount of distortion imparted by one or more optical elements of an image viewer of a computer-assisted surgical system on images displayed by way of the image viewer. Operation 702 may be performed in any of the ways described herein.

At operation 704, the image processing system obtains image data representative of an image that is displayable by way of the image viewer. Operation 704 may be performed in any of the ways described herein.

At operation 706, the image processing system modifies, based on the distortion factor, the image data to correct for the distortion imparted by the one or more optical elements of the image viewer, the modifying resulting in modified image data representative of the image. Operation 706 may be performed in any of the ways described herein.

At operation 708, the image processing system directs the computer-assisted surgical system to display, based on the modified image data, the image by way of the image viewer. Operation 708 may be performed in any of the ways described herein.

Although the preceding disclosure describes correcting for distortion imparted by one or more optical elements of an image viewer on images captured by an imaging device, it is understood that the distortion correction concepts described herein may be applied to any type of content or combination thereof that is displayable by way of an image viewer. For example, distortion correction may be applied to one or more images and/or other content that are not captured by an imaging device such as computer-generated renderings, augmented reality content, virtual reality content, user interface elements, text, graphics, images and/or video supplied by a third party (e.g., a customer), and/or any other type of content.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Illustrative non-volatile storage media include, but are not limited to, read-only memory, flash memory, a solid-state drive, a magnetic storage device (e.g., a hard disk, a floppy disk, magnetic tape, etc.), ferroelectric random-access memory (“RAM”), and an optical disc (e.g., a compact disc, a digital video disc, a Blu-ray disc, etc.). Illustrative volatile storage media include, but are not limited to, RAM (e.g., dynamic RAM).

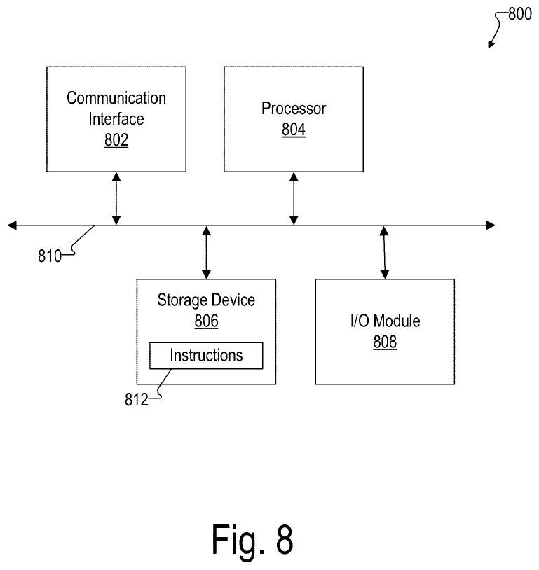

FIG. 8 illustrates an example computing device 800 that may be specifically configured to perform one or more of the processes described herein. As shown in FIG. 8, computing device 800 may include a communication interface 802, a processor 804, a storage device 806, and an input/output (“I/O”) module 808 communicatively connected one to another via a communication infrastructure 810. While an example computing device 800 is shown in FIG. 8, the components illustrated in FIG. 8 are not intended to be limiting. Additional or alternative components may be used in other embodiments. Components of computing device 800 shown in FIG. 8 will now be described in additional detail.

Communication interface 802 may be configured to communicate with one or more computing devices. Examples of communication interface 802 include, without limitation, a wired network interface (such as a network interface card), a wireless network interface (such as a wireless network interface card), a modem, an audio/video connection, and any other suitable interface.

Processor 804 generally represents any type or form of processing unit capable of processing data and/or interpreting, executing, and/or directing execution of one or more of the instructions, processes, and/or operations described herein. Processor 804 may perform operations by executing computer-executable instructions 812 (e.g., an application, software, code, and/or other executable data instance) stored in storage device 806.

Storage device 806 may include one or more data storage media, devices, or configurations and may employ any type, form, and combination of data storage media and/or device. For example, storage device 806 may include, but is not limited to, any combination of the non-volatile media and/or volatile media described herein. Electronic data, including data described herein, may be temporarily and/or permanently stored in storage device 806. For example, data representative of computer-executable instructions 812 configured to direct processor 804 to perform any of the operations described herein may be stored within storage device 806. In some examples, data may be arranged in one or more databases residing within storage device 806.

I/O module 808 may include one or more I/O modules configured to receive user input and provide user output. I/O module 808 may include any hardware, firmware, software, or combination thereof supportive of input and output capabilities. For example, I/O module 808 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 808 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 808 is configured to provide graphical data to a display for presentation to a user. The graphical data may be representative of one or more graphical user interfaces and/or any other graphical content as may serve a particular implementation.

In some examples, any of the systems, computing devices, and/or other components described herein may be implemented by computing device 800. For example, processor 302 may be implemented by processor 804.

In the preceding description, various example embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
one or more processors configured to perform a process comprising:
obtaining a distortion factor representative of an amount of distortion imparted by one or more optical elements of an image viewer of a computer-assisted surgical system on images displayed by way of the image viewer;
obtaining image data representative of an image that is displayable by way of the image viewer;
modifying, based on the distortion factor, the image data to correct for the distortion imparted by the one or more optical elements of the image viewer, the modifying resulting in modified image data representative of the image; and
directing the computer-assisted surgical system to display, based on the modified image data, the image by way of the image viewer.

2. The system of claim 1, wherein the modifying of the image data includes pre-distorting pixel values of a subset of pixels included in a plurality of pixels of the image.

3. The system of claim 1, wherein the one or more processors include a field programmable gate array (FPGA).

4. The system of claim 3, wherein the obtaining of the distortion factor includes accessing a pre-programmed distortion factor from logic of the FPGA.

5. The system of claim 1, wherein the obtaining of the distortion factor includes generating the distortion factor based on one or more factors associated with operation of the image viewer.

6. The system of claim 1, wherein the process further comprises:
detecting a change in one or more factors associated with operation of the image viewer;
obtaining, based on the change in the one or more factors, an updated distortion factor; and
modifying, based on the updated distortion factor, the image data to correct for the distortion imparted by the one or more optical elements of the image viewer.

7. The system of claim 1, wherein:
the image is captured by an imaging device communicatively coupled to the computer-assisted surgical system; and
a latency from image capture to displaying the image by way of the image viewer is less than 50 milliseconds.

8. A computer-assisted surgical system comprising:
an image viewer including:
a display device; and
an optical assembly through which a user views images displayed by the display device, the optical assembly provided along an optical path between the display device and an eye of the user, the optical assembly imparting a distortion on images displayed by the display device when viewed through the optical assembly;
wherein the display device is configured to:
receive images to be displayed by way of the image viewer; and
display the images based on a distortion factor that corrects for the distortion imparted by the optical assembly.

9. The computer-assisted surgical system of claim 8, wherein the image viewer further includes one or more processors configured to modify the images to be displayed by way of the image viewer device based on the distortion factor.

10. The computer-assisted surgical system of claim 9, wherein a latency associated with the one or more processors modifying the images and the displaying of the images is less than 50 milliseconds.

11. The computer-assisted surgical system of claim 9, wherein the one or more processors include a field programmable gate array (FPGA).

12. The computer-assisted surgical system of claim 8, wherein the display device includes pixels that are arranged in an array based on the distortion factor to correct for the distortion imparted by the optical assembly.

13. The computer-assisted surgical system of claim 12, wherein an arrangement of the pixels of the display device is non-rectilinear.

14. The computer-assisted surgical system of claim 8, further comprising one or more reflectors provided along the optical path between the display device and the optical assembly that change a direction of the optical path.

15. The computer-assisted surgical system of claim 8, further comprising:
one or more manipulator arms configured to hold an instrument; and
one or more actuators for controlling the one or more manipulator arms.

16. The computer-assisted surgical system of claim 15, wherein:
the images are captured by an imaging device communicatively coupled to the computer-assisted surgical system; and
the imaging device is engaged with a manipulator arm included in the one or more manipulator arms.

17. The computer-assisted surgical system of claim 15, wherein the image viewer is part of a surgeon console that is communicatively coupled to the one or more manipulator arms and the imaging device.

18. A method comprising:
obtaining, by an image processing system, a distortion factor representative of an amount of distortion imparted by one or more optical elements of an image viewer of a computer-assisted surgical system on images displayed by way of the image viewer;
obtaining, by the image processing system, image data representative of an image that is displayable by way of the image viewer;
modifying, by the image processing system and based on the distortion factor, the image data to correct for the distortion imparted by the one or more optical elements of the image viewer, the modifying resulting in modified image data representative of the image; and
directing, by the image processing system, the computer-assisted surgical system to display, based on the modified image data, the image by way of the image viewer.

19. The method of claim 18, wherein the modifying of the image data includes pre-distorting pixel values of a subset of pixels included in a plurality of pixels of the image.

20. The method of claim 18, further comprising:
detecting, by the image processing system, a change in one or more factors associated with operation of the image viewer;
obtaining, by the image processing system and based on the change in the one or more factors, an updated distortion factor; and
modifying, by the image processing system and based on the updated distortion factor, the image data to correct for the distortion imparted by the one or more optical elements of the image viewer.
